(19) United States
(12) Patent Application Publication
(10) Pub. No.: US 2003/0170848 A1
(43) Pub. Date: Sep. 11, 2003

(54) N-methyltransferase

(76) Inventor: Bala Rathinasabapathi, Gainesville, FL (US)

Correspondence Address: Stanley A. Kim, Ph.D., Esq., Akerman, Senterfitt & Eidson, P.A., 222 Lakeview Avenue, Suite 400, P.O. Box 3188, West Palm Beach, FL, 33402-3188, US

(21) Appl. No.: 10/132,100

(22) Filed: Apr. 24, 2002

Related U.S. Application Data
(60) Provisional application No. 60/286,162, filed on Apr. 24, 2001.

(51) Int. Cl. G01N 33/53; G01N 33/537; G01N 33/543; C07H 21/04; C12N 9/10; C12P 21/02; C12N 5/06

(52) U.S. Cl. 435/193; 530/388.26; 435/7.92; 435/69.1; 435/320.1; 435/325; 536/23.2

(57) ABSTRACT

A β-alanine N-methyltransferase was isolated from L. latifolium. The purified enzyme catalyzes the N-methylation of β-ala betaine, has an isoelectric point of about 5.15 and an apparent molecular weight of about 43 kilodaltons. The purified enzyme was partially sequenced. The purified enzyme or portions thereof can be used to make antibodies that specifically bind the enzyme, and can be introduced into a cell to modulate the cell's N-methyltransferase activity level and ability to resist environmental stress.

### CROSS REFERENCE TO RELATED APPLICATION

The present application claims the priority of U.S. provisional patent application No. 60/286,162, filed Apr. 24, 2001, and entitled “N-Methyltransferase Involved In Beta-alanine Betaine Synthesis.”

### STATEMENT AS TO FEDERALLY SPONSORED RESEARCH

The invention was made with U.S. government support under grant numbers NRICGP 2001-35318-10947 and HOS3807, both awarded by the U.S. Department of Agriculture. The U.S. government may have certain rights in the invention.

### FIELD OF THE INVENTION

The invention relates generally to the fields of biology, botany, and agricultural sciences. More particularly, the invention relates to the purification, cloning and characterization of an N-methyl transferase from Limonium latifolium, and to methods and compositions for modulating a plant's resistance to environmental stress.

### BACKGROUND

Many plants, bacteria and marine algae accumulate quaternary ammonium compounds (QACs) in response to abiotic stresses such as drought and salinity. Gorham J (1995) Betaines in higher plants—biosynthesis and role in stress metabolism. In R M Wallsgrove, ed, Amino acids and their derivatives in higher plants. Cambridge University Press, Cambridge, pp 173-203. QACs can accumulate to high concentrations to increase the osmotic pressure of the cytoplasm without perturbing metabolism. Yancey P H (1994) Compatible and counteracting solutes. In K. Strange, ed, Cellular and molecular physiology of cell volume regulation. CRC Press, Boca Raton, Fla. pp 81-109 They also stabilize enzymes and membranes. Id. The synthetic pathway to glycine betaine, the most common QAC, has therefore been the target of recent metabolic engineering efforts to improve plant stress tolerance. McNeil et al. (1999) Plant Physiol 120:945-949; Rathinasabapathi (2000) Ann Bot 86:709-716; Sakamoto and Murata (2000) J Exp Bot 51:81-88. However, these efforts have met with only limited success due to metabolic constraints on the availability of the precursor choline. Hayashi et al. (1997) Plant J 12:133-142; Nuccio et al. (1998) Plant J 12:133-142; Huang et al. (2000) Plant Physiol 122: 747-756.

Most members of the highly stress-tolerant plant family Plumbaginaceae accumulate β-alanine (β-ala) betaine instead of glycine betaine. Hanson et al. (1991) Plant Physiol 97:1199-1205; Hanson et al. (1994) Proc Natl Acad Sci USA 91:306-310. It was proposed that β-ala betaine is a more suitable osmoprotectant than glycine betaine under saline hypoxic conditions since the first step in glycine betaine synthesis requires molecular oxygen. Id. Further, β-ala betaine accumulation was proposed to be an evolutionary strategy to avoid metabolic limitations for choline (Hanson et al., 1994) since β-ala betaine is synthesized from the ubiquitous primary metabolite β-alanine.

To further investigate the synthesis and biological significance of β-ala betaine, radiotracer experiments were conducted. These experiments showed that β-ala betaine is synthesized by S-adenosyl-L-methionine (AdoMet) dependent N-methylation of β-alanine via N-methyl β-alanine and N,N-dimethyl β-alanine (Rathinasabapathi et al., 2000; FIG. 1). Using a rapid and sensitive radiometric assay, AdoMet dependent N-methyltransferase (NMTase) activities were demonstrated in β-ala betaine accumulating members of the Plumbaginaceae family (Rathinasabapathi et al., 2000). Heretofore, however, the protein responsible for the NMTase activities was uncharacterized.

### SUMMARY

The invention relates to the purification and characterization of an NMTase from L. latifolium. The NMTase was purified from L. latifolium leaf tissue using a seven-step protocol. Biochemical characterization of the purified enzyme indicated that it had an isoelectric point (pI) of 5.1, and that it was a dimer of 43 kD subunits. Functional studies indicated that the purified enzyme catalyzes all three of the N-methylations involved in the synthesis of β-ala betaine. Peptide sequencing studies indicated that the purified NMTase shared some sequence similarity to methyltransferases from other organisms.

Accordingly, the invention features a purified N-methyltransferase that is present in L. latifolium. The purified N-methyltransferase has an isoelectric point of about 5.15 and migrates on SDS-PAGE at about 43 kilodaltons. The N-methyltransferase can include one or more of the amino acid sequences listed herein as SEQ ID NOs: 1-5. Also within the invention are purified proteins that include the amino acid sequence of SEQ ID NOs: 1, 2, 3, 4, or 5.

In another aspect, the invention features a purified antibody that specifically binds an N-methyltransferase present in L. latifolium and/or a polypeptide made up of an amino acid sequence of SEQ ID NOs: 1, 2, 3, 4, or 5.

Also featured in the invention is a cell into which has been introduced a purified N-methyltransferase that is present in L. latifolium; has an isoelectric point of about 5.15; and/or migrates on SDS-PAGE at about 43 kilodaltons. Another cell within the invention is one into which has been introduced the amino acid sequence of: SEQ ID NOs: 1, 2, 3, 4 or 5. Cells of the invention can be plant cells such as those in a plant.

Unless otherwise defined, all technical terms used herein have the same meaning as commonly understood by one of ordinary skill in the art to which this invention belongs. Definitions of molecular biology terms can be found, for example, in Rieger et al., Glossary of Genetics: Classical and Molecular, 5th edition, Springer-Verlag: New York, 1991; and Lewin, Genes V, Oxford University Press: New York, 1994.

The terms “isolated” and “purified,” as used herein with respect to an enzyme, refer to a enzymatically active molecule substantially separated from other molecules that are present in a cell or organism in which the enzymatically active molecule naturally occurs. A purified NMTase includes, e.g., a NMTase-containing cell extract that has been subjected to one or more number of chromatographic separations. The terms “isolated” and “purified” as used herein also refer to a molecule produced artificially (i.e., outside the organism in which the molecule naturally occurs) by molecular biological techniques (e.g., recombinant DNA technology) or chemical synthesis (e.g., peptide synthesis).

As used herein, “protein” or “polypeptide” are used synonymously to mean any peptide-linked chain of amino acids, regardless of length or post-translational modification, e.g., glycosylation or phosphorylation. A “purified” polypeptide is one that has been substantially separated or isolated away from other polypeptides in a cell or organism in which the polypeptide naturally occurs (e.g., 30, 40, 50, 60, 70, 80, 90, 95, 96, 97, 98, 99, 100% free of contaminants).

The term “antibody” means an immunoglobulin or fragment of an immunoglobulin that retains a function of an intact immunoglobulin, e.g., antigen-binding or effector functions.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

The above and further advantages of this invention may be better understood by referring to the following description taken in conjunction with the accompanying drawings, in which:

FIG. 1 is a schematic overview of the synthetic pathway to β-ala betaine. Each downward arrow represents an AdoMet dependent N-methylation step.

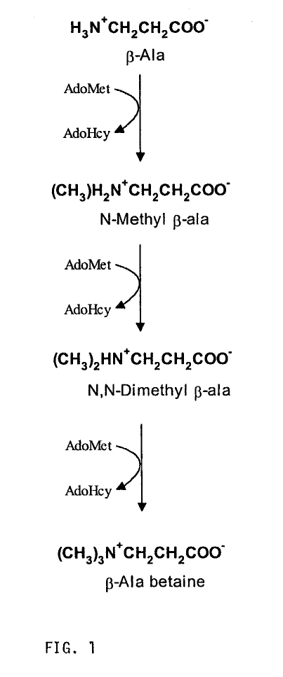

FIG. 2 is a graph showing the NMTase activity and protein amounts in fractions separated by anion exchange chromatography using DEAE-Fractogel. The procedure is described in the methods section. NMTase activities (nmol h⁻¹/fraction) against β-alanine (BA), N-methyl β-alanine (MM) and N, N-dimethyl β-alanine (DM) are indicated by squares, triangles and stars, respectively. The predicted KCl gradient (20 to 300 mM) is shown in a dotted line. Protein content (mg/fraction), estimated by the modified Lowry's method (Peterson et al., Anal Biochem 83:346-356, 1977) is shown in open circles.

FIG. 3 is a graph showing the NMTase activity and protein concentrations in fractions separated by N,N-dimethyl β-alanine substrate affinity column chromatography. Protein elution profile by OD₂₈₀ is shown for the unbound fraction (UB) and elutions (KW=50 mM KCl wash, SE=substrate elution and KE=200 mM KCl elution). NMTase activities (nmol h⁻¹/fraction) with β-alanine (BA), N-methyl β-alanine (MM) and N,N-dimethyl β-alanine (DM) measured in the wash and the elutions are shown in the inset.

FIG. 4 is a graph showing the NMTase activity and protein concentrations in fractions separated by adenosine agarose affinity chromatography. Protein elution profile by OD₂₈₀ of fractions is shown for unbound (UB), 200 mM KCl wash (KW) and substrate elution with 5 mM AdoMet (AE). Note that absorbance in the AdoMet elution is largely due to the AdoMet and not protein. NMTase activities (nmol h⁻¹/fraction) with β-alanine (BA), N-methyl β-alanine (MM) and N,N-dimethyl β-alanine (DM) measured in the unbound fraction, 200 mM KCl wash and AdoMet elution are shown in the inset.

FIG. 5 is an autoradiograph of a gel from SDS-PAGE analysis of the purified L. latifolium NMTase and Photoaffinity labeling. Lane A. Precision SDS-Protein markers (Bio-Rad 161-0362). Lane B. SDS-Denatured protein (20 ng) from the adenosine agarose step (Table I), separated in a 12% acrylamide gel and stained with silver stain. Lane C. Partially purified (100-fold) NMTase fraction following photoaffinity labeling with S-Adenosyl-L-[methyl-³H]Met, SDS-PAGE and autoradiography. Lane D. Partially purified (100-fold) NMTase fraction following photoaffinity labeling with S-Adenosyl-L-[methyl-³H]Met in the presence of AdoHCy, SDS-PAGE and autoradiography.

FIG. 6 illustrates two graphs showing the results of a kinetic analysis of L. latifolium NMTase protein. (A) Effect of varying Ado-Met concentration on the reaction velocity shown in a plot of s/v versus s. Ado-Met concentration was varied from 0 to 300 μM and β-alanine concentration was kept at 10 mM. Inset shows the direct plot. (B) Effect of varying β-alanine on the reaction velocity shown in a plot of s/v versus s. β-alanine levels were varied between 0 and 10 mM. Ado-Met concentration was kept at 100 μM.

### DETAILED DESCRIPTION

The below described preferred embodiments illustrate adaptations of these compositions and methods. Nonetheless, from the description of these embodiments, other aspects of the invention can be made and/or practiced based on the description provided below.

### Purified β-alanine NMTase Polypeptides

The present invention provides a purified β-alanine NMTase polypeptide isolated from L. latifolium. As described in the Examples section below, a β-alanine N-methyltransferase was isolated from L. latifolium using a series of purification steps. This protein was characterized both physically and functionally. Isoelectric focusing analysis showed that the purified enzyme exhibited an isoelectric point of 5.15. SDS-PAGE analysis showed that the purified enzyme migrated at about 43 kD. Functionally, the purified enzyme was capable of methylating β-alanine, N-methyl β-alanine, and N,N-dimethyl β-alanine.

In addition to the whole β-alanine NMTase polypeptide, the invention also provides fragments of the enzyme. Fragments of the enzyme can be made by treating the whole β-alanine NMTase polypeptide with one or more proteases, or by subjecting it to one of the techniques described below in the examples section. Peptide sequencing revealed the amino acid sequence of 5 oligopeptides (SEQ ID NOs: 1-5) making up parts of the whole β-alanine NMTase polypeptide. Thus, the invention also provides purified polypeptides including one or more of these sequences. Fragments of the whole β-alanine NMTase polypeptide can be made by chemically synthesizing the oligopeptides by known techniques.

### Anti-β-alanine NMTase Antibodies

β-alanine NMTase polypeptides (or immunogenic fragments or analogs thereof) can be used to raise antibodies useful in the invention. Such polypeptides can be isolated as described herein. Fragments of β-alanine NMTase can be prepared by digesting the native protein with proteases or by synthesizing oligopeptides based on known amino acid sequence information. In general, β-alanine NMTase polypeptides can be coupled to a carrier protein, such as KLH, as described in Ausubel et al., supra, mixed with an adjuvant, and injected into a host mammal. Antibodies produced in that animal can then be purified by peptide antigen affinity chromatography. In particular, various host animals can be immunized by injection with a β-alanine NMTase polypeptide or an antigenic fragment thereof. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Other potentially useful adjuvants include BCG (bacille Calmette-Guerin) and Corynebacterium parvum.

Polyclonal antibodies are heterogeneous populations of antibody molecules that are contained in the sera of the immunized animals. Antibodies within the invention therefore include polyclonal antibodies and, in addition, monoclonal antibodies, single chain antibodies, Fab fragments, F(ab′)₂ fragments, and molecules produced using a Fab expression library. Monoclonal antibodies, which are homogeneous populations of antibodies to a particular antigen, can be prepared using the β-alanine NMTase polypeptides described above and standard hybridoma technology (see, for example, Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, N.Y., 1981; Ausubel et al., supra). In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. A hybridoma producing a mAb of the invention may be cultivated in vitro or in vivo. The ability to produce high titers of mAbs in vivo makes this a particularly useful method of production.

Once produced, polyclonal or monoclonal antibodies can be tested for specific β-alanine NMTase recognition by Western blot or immunoprecipitation analysis by standard methods, for example, as described in Ausubel et al., supra. Antibodies that specifically recognize and bind to β-alanine NMTase are useful in the invention. For example, such antibodies can be used in an immunoassay to monitor the level of β-alanine NMTase produced by a plant (e.g., to determine the amount or subcellular location of β-alanine NMTase).

In some cases it may be desirable to minimize the potential problems of low affinity or specificity of antisera. In such circumstances, two or three fusions can be generated for each protein, and each fusion can be injected into at least two rabbits. Antisera can be raised by injections in a series, preferably including at least three booster injections. Antiserum is also checked for its ability to immunoprecipitate recombinant β-alanine NMTase polypeptides or control proteins, such as glucocorticoid receptor, CAT, or luciferase.

The antibodies of the invention can be used, for example, in the detection of β-alanine NMTase in a biological sample. Antibodies also can be used in a screening assay to measure the effect of a candidate compound on expression or localization of β-alanine NMTase. Additionally, such antibodies can be used to interfere with the interaction of β-alanine NMTase and other molecules that interact with β-alanine NMTase.

Techniques described for the production of single chain antibodies (U.S. Pat. Nos. 4,946,778, 4,946,778, and 4,704,692) can be adapted to produce single chain antibodies against a β-alanine NMTase polypeptide, or a fragment thereof. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed (Huse et al., Science 246:1275, 1989) to allow rapid and easy identification of monoclonal Fab fragments with the desired specificity.

### A Cell into Which Has Been Introduced a Purified β-alanine NMTase

The invention also provides a cell into which has been introduced a purified N-methyltransferase or fragment thereof. The whole enzyme or a portion thereof (e.g., a protein that includes one of SEQ ID NOs: 1-5) can be introduced into a cell by any known technique. For example, the purified enzyme can be introduced into a cell by microinjection. Introduction of the enzyme into a cell can modulate the methylation of substrates such as β-alanine, N-methyl β-alanine, and N,N-dimethyl β-alanine. Such a modulation is expected to be useful in increasing or reducing stress tolerance in the cell. The cell into which has been introduced a purified N-methyltransferase or fragment thereof is preferably a plant cell, e.g. one other than L. latifolium. The plant cell can be one within a plant.

### Detection of β-alanine NMTase

The invention encompasses methods for detecting the presence of β-alanine NMTase protein in a biological sample as well as methods for measuring the level of β-alanine NMTase protein in a biological sample. Such methods are useful for examining plant intracellular signaling pathways associated with stress resistance.

An exemplary method for detecting the presence or absence of β-alanine NMTase in a biological sample involves obtaining a biological sample from a test plant (or plant cell) and contacting the biological sample with a compound or an agent capable of detecting a β-alanine NMTase polypeptide. A preferred agent for detecting a β-alanine NMTase polypeptide is an antibody capable of binding to a β-alanine NMTase polypeptide, preferably an antibody with a detectable label. Such antibodies can be polyclonal, or more preferably, monoclonal. An intact antibody, or a fragment thereof (e.g., Fab or F(ab′)₂) can be used.

Detection methods of the invention can be used to detect a β-alanine NMTase polypeptide in a biological sample in vitro as well as in vivo. For example, in vitro techniques for detecting a β-alanine NMTase polypeptide include enzyme-linked immunosorbent assays (ELISAs), Western blots, immunoprecipitations and immunofluorescence. Furthermore, in vivo techniques for detection of a β-alanine NMTase polypeptide include introducing into a plant or plant cell labeled anti-β-alanine NMTase antibody. For example, the antibody can be labeled with a radioactive marker whose presence and location in a plant can be detected by standard imaging techniques.

### EXAMPLES

The present invention is further illustrated by the following specific examples. The examples are provided for illustration only and are not to be construed as limiting the scope or content of the invention in any way.

### Example 1

### Materials and Methods

Chemicals. If not otherwise indicated, chemicals used were from Sigma Chemical Co (St. Louis, Mo.) and were of the highest purity available. Amberlite XAD-4 resin beads (Aldrich, Milwaukee, Wis.) were washed in 20 column volumes each of methanol and water and stored in water at 4° C. until use. S-Adenosyl-L-[methyl-³H]Met was purchased from NEN Life Science Products (Boston, Mass.) at a specific activity of 82 Ci mmol⁻¹ (3 TBq mmol⁻¹) and used without further purification. S-Adenosyl-L-Met, chloride salt was purified using Whatman CM 52 ion exchange chromatography according to Chirpich (1968) Lysine-2,3-aminomutase: purification and properties. Ph.D. thesis. University of California, Berkeley. N-methyl β-alanine and N,N-dimethyl β-alanine were synthesized as described previously (Rathinasabapathi et al., Ann Bot 86:709-716, 2000). N,N-dimethyl β-alanine sepharose 4B affinity resin was prepared by coupling the amino group of 1,6 diaminohexane in EAH-sepharose (Amersham-Pharmacia Biotech, Piscataway, N.J.) to the carboxyl group of N,N-dimethyl β-alanine, using a carbodiimide procedure (Hoare and Datta, Arch Biochem Biophys 277:122-129, 1990). Adenosine agarose affinity resin was prepared from 5′-AMP-agarose by the method of James et al. (J Biol Chem 270:22344-22350, 1995).

Plant Material. Seeds of L. latifolium (Sm.) O. Kuntze, were from Park Seed Co (Greenwood, S.C.). Plants were grown in Metro-Mix 200 (Scotts-Sierra, Marysville, Ohio) in wooden boxes (2 ft×2 ft×8 inches deep) in a greenhouse in Gainesville, Fla. The plants were fertilized once a week using a 200 ppm solution of a fertilizer (N:P:K 20:20:20). Other species of Limonium might also be used in the invention as a source of the NMTase.

Enzyme Extraction. Fully expanded leaves were harvested, briefly washed in a mild soap solution and rinsed in de-ionized water prior to extraction. Leaves were sliced into about 1 cm wide strips, frozen in liquid nitrogen and ground to a powder in a mortar. The powder was transferred to a blender containing freshly prepared extraction medium, 400 mL per 100 g fresh weight leaves. The extraction medium contained the following in 0.1 M Tris-HCl pH 8: 0.2 M sodium tetraborate, 2 mM DTT, 5 mM EDTA, 10% (v/v) glycerol, 4% (w/v) insoluble PVPP, 6% (w/v) Amberlite XAD-4, 10 μM leupeptin, 0.2 mM AEBSF, 1 μM pepstatin A, 1 μM Bestatin, 1 μM E-64 and 1 mM 1,10-phenanthroline. The tissue was blended in the extraction buffer for 3 min at maximum speed, filtered through four layers of autoclaved cheesecloth and centrifuged at 20,000 g for 30 min in a refrigerated centrifuge (model J2-HS, Beckman Instruments, Fullerton, Calif.). The supernatant (crude extract) was saved for further purification (see below). An aliquot of the crude extract was desalted by passage through Sephadex G-25 columns (PD10, Amersham Pharmacia, Piscataway, N.J.) prior to assays for total protein and NMTase activities.

Enzyme Assay. The NMTase activities with β-alanine, N-methyl β-alanine and N,N-dimethyl β-alanine were assayed using a radiometric method (Rathinasabapathi et al., Physiol Plant 109:225-231, 2000), with modifications as stated below. The assay mixture contained 54 μL of enzyme preparation in a total volume of 100 μL containing 0.1 M Tris-HCl buffer pH 8.0, 2 mM DTT, 10 mM methyl acceptor, 100 μM AdoMet and 0.027 μM S-Adenosyl-L-[methyl-³H]Met (200 nCi of radioactivity). Following incubation at 30° C. for 30 minutes, the reactions were stopped by the addition of 10 μL of 10% (w/v) trichloroacetic acid containing 1 mM of methylated products as unlabeled carrier. Activated charcoal (38 mg.ml⁻¹) in 0.1 N acetic acid, 250 μL per assay, was added and centrifuged for five minutes. The radioactive product in the supernatant was quantified in 75% Ready Gel using a liquid scintillation counter (Beckman Instruments, Fullerton, Calif.). The counting efficiency was 30%.

Enzyme Purification. All protein purification steps were done at 4° C. For column chromatography steps, a low pressure column chromatography system (Bio-Rad, Hercules, Calif.) consisting of a peristaltic pump, UV monitor, a fraction collector and a chart recorder was used. All columns were equilibrated in buffer A (20 mM Tris-HCl pH 8.0, 10% glycerol and 2 mM DTT), prior to use. If required, protein preparations between purification steps were concentrated using a 10 kD cut-off Centriprep (Millipore, Mass.) centrifugal filter device. Protein precipitating between 10% (w/v) and 15% (w/v) PEG 8000 (Fisher Biotech, Fair Lawn, N.J.) was dissolved in buffer A. The NMTase activities were stable in this fraction for at least two months when stored at −80° C. For heat treatment, 25 mL of the PEG-precipitated protein dissolved in buffer A was exposed to 50° C. in a water bath for 15 min. The preparation then was centrifuged at 20,000 g for 20 min and the supernatant was collected. For anion exchange chromatography, protein (about 40 to 50 mg) from the heat treatment step was loaded onto a column (13.5 cm×3 cm ) containing 50 mL DEAE-Fractogel EMD ion exchanger (EM Separations Technology, Gibbstown, N.J.). The column was washed with 50 mL buffer A and then with 90 mL buffer A containing 20 mM KCl. The bound proteins were then eluted from the column with 104-mL linear 20 mM to 300 mM KCl gradient in buffer A containing 0.1 mM AEBSF. Fractions (7.5 mL) were collected and assayed for NMTase activities and protein. Fractions with specific activities equal to and above that of the load were pooled and concentrated to 1 to 2 mL prior to gel filtration. Gel filtration was performed on a 70 cm×1.7 cm Sephacryl S-200 HR column (Amersham Pharmacia, Piscataway, N.J.). Fractions (3 mL each) were assayed for NMTase activities and protein, and those with specific activities equal to or above that of the load were pooled. The pooled fractions from the gel filtration step were loaded onto a N,N-dimethyl β-alanine-EAH Sepharose 4B affinity column (5 cm×0.8 cm i.d., 2 mL). The column was washed with buffer A, and with 50 mM KCl. The bound proteins were eluted using buffer A containing 10 mM each of β-alanine and N,N-dimethyl β-alanine and using buffer A containing 200 mM KCl. Substrate elution and the 200 mM KCl elution were pooled and concentrated to 1.3 mL before being loaded on to a continuous electrophoresis prep cell (Model 491, Bio-Rad, Hercules, Calif.). The prep cell used a native-gel column made up of 40 mL of 6% (w/v) acrylamide in 24 mM Tris-CAPS buffer, pH 9.3 (McLellan, 1982). Electrophoresis was at 300 V for 2 h with 24 mM Tris-CAPS buffer, pH 9.3 and the proteins were eluted with buffer A. Fractions (3 mL each) were assayed for NMTase activities and protein. Fractions with specific activities equal to and above that of the load were pooled, concentrated and loaded onto an adenosine agarose affinity gel (3 mL column). Non-specific proteins were washed off the column with buffer A containing 0.2 M KCl and the bound proteins were eluted with 5 mM AdoMet and 0.2 M KCl in buffer A. The eluate was concentrated prior to NMTase and protein assays.

Estimation of Native Molecular Weight. Gel filtration was performed using Sephacryl S-200 column chromatography as described above. The column was calibrated with marker proteins alcohol dehydrogenase (150 kD), bovine serum albumin (66 kD), ovalbumin (45 kD) and cytochrome C (12.4 kD).

Estimation of Protein. Protein was estimated after precipitating it from appropriate volumes of fractions using Lowry's method as modified by Peterson (Anal Biochem 83:346-356, 1977). Bovine serum albumin was used as the standard.

SDS-PAGE. SDS-PAGE was performed according to Laemmli (Nature 227:680-685, 1970) in 12% (w/v) separation gel and 5% (w/v) stacking gel. Proteins were visualized with Coomassie Brilliant Blue or silver-stain.

Estimation of pI. A protein fraction purified about 10-fold was subjected to isoelectric focusing in an IsoGel agarose IEF plate pH 3 to 10 system (FMC Bioproducts, Rockland, Me.) at 1000 V for 40 min. The anolyte was 0.5 M acetic acid pH 2.6 and the catholyte was 1 M NaOH, pH 13. Two lanes in the IEF plate were stained with Coomassie Blue to visualize the proteins and the rest of the agarose gel was sliced into 2 mm strips and assayed for NMTase activities. Maximum activities against all the three methyl acceptors corresponded to pH 5.15 in a standard curve of pIs for known standard proteins focused in the same IEF plate.

Photoaffinity Labeling. To identify the protein subunit(s) binding to AdoMet, photoaffinity labeling (Som and Friedman, J Biol Chem 265:4278-4283, 1990) was done on protein samples at various stages of purification from the ion exchange chromatography stage onward using the method as described by Smith et al. (Physiol Plant 108:286-294, 2000).

Kinetic characterization. A partially-purified enzyme preparation after the anion exchange column chromatography step (Table I) was used. The activity was stable in this fraction when stored at −80° C. for up to two months. The assay procedure and conditions were similar to that described above except that the duration of the assay was reduced to 20 min and the substrate concentrations were varied as indicated. The enzyme concentration employed (15 μg protein per assay) gave a linear reaction velocity during the incubation period. Kinetic constants were derived from the X and Y intercepts of a linear plot of s/v versus s drawn from triplicate assay results (Henderson, P. J. F., Statistical analysis of enzyme kinetic data. In R. Eisenthal, M J Danson, eds, Enzyme assays a practical approach, IRL Press, Oxford, pp. 276-316 1993). The experiment was repeated twice with similar results.

Effect of a Thiol Reagent. Protein purified using PEG precipitation was assayed with or without added DTT in the presence and absence of the thiol reagent p-hydroxymercuribenzoic acid.

Peptide Sequencing. Purified NMTase (400 ng) was separated by SDS-PAGE and stained with Coomassie R-250 and destained in 10% (v/v) methanol and 5% (v/v) acetic acid. The band of protein was digested by endoproteinase Lys-C, separated in an HPLC and sequenced using Edman degradation (Rosenberg I. M., Peptide mapping and microsequencing. In Protein analysis and purification. Birkhauser, Boston, pp. 183-206, 1996). The peptide sequences were compared to other proteins in the databases using the BLAST program (Altschul et al., Nucleic Acids Res 25:3389-3402, 1997).

### Example 2

### Results

Peptide Studies. Because L. latifolium leaves are rich in phenolics, the enzyme purification protocol employed nonionic polymeric adsorbent XAD4, polyvinyl polypyrrolidone PVPP (Loomis, Methods in Enzymol 31:528-544, 1974), and protease inhibitors in the extraction medium and elution buffers used in early chromatography steps. A series of steps were employed to purify the NMTase as detected by assays with β-alanine, N-methyl β-alanine and N,N-dimethyl β-alanine (Table I). Each step was found to improve NMTase specific activities in smaller scale trials. However, when scaled up, certain steps did not reproducibly improve purity (Table I, heating and Sephacryl S-200 column chromatography for example).

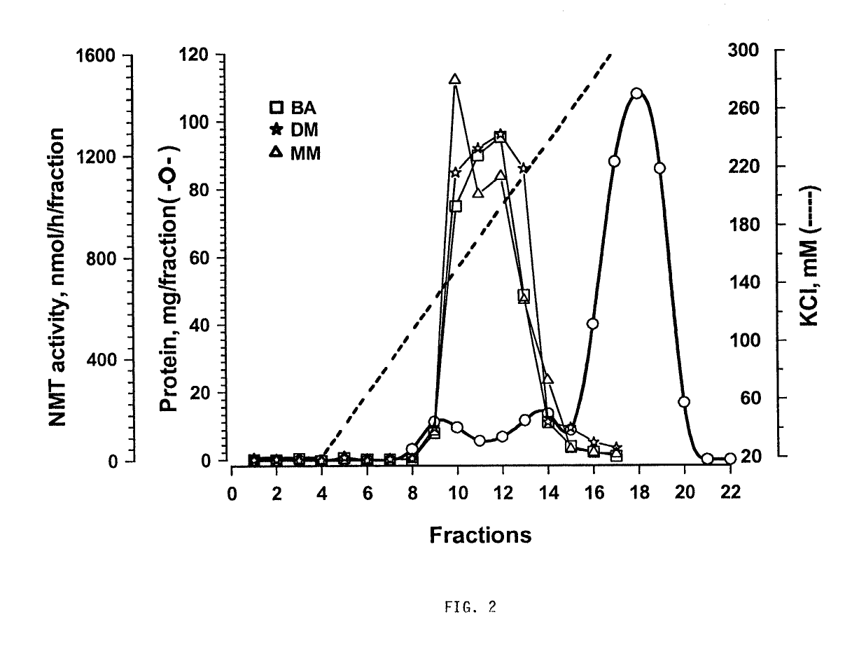

PEG precipitation step was employed primarily to concentrate the extracted protein in a stable form, achieving a 2-fold purification. In separate trials, heat treatment of the PEG fraction resulted in 2-fold improvement in specific activities. DEAE-Fractogel anion exchange column chromatography improved specific activities to about 6-fold (Table I) as shown in FIG. 2. NMTase activities eluted from DEAE-fractogel column between 125 mM and 200 mM KCl, ahead of the majority of proteins (FIG. 2).

Following anion exchange chromatography, the protein fraction was purified by gel filtration chromatography on Sephacryl S-200. NMTase activity eluted as a single peak with an elution volume corresponding to a native molecular weight of 80 kD. The use of protease inhibitors proved extremely valuable in this step. Without inhibitors, NMTase activity eluted in four peaks corresponding to 110, 80, 40 and 20 kD, the 80 kD NMTase being more than 50% of the total recovered activity, and the total activity recovered was substantially reduced. Activity at 110 kD was probably due to protein aggregation.

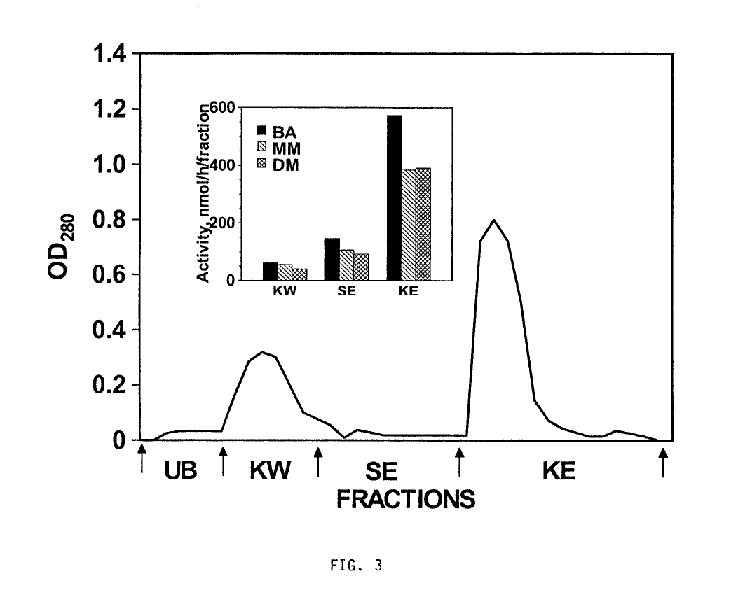

N,N-Dimethyl β-alanine-EAH sepharose affinity matrix bound most proteins loaded (FIG. 3). β-alanine and N,N-Dimethyl β-alanine at 10 mM each were not sufficient to elute most NMTase activities from this column. Elution with 200 mM KCl was more effective (FIG. 3, inset), suggesting that the matrix also had anion exchange characteristics in addition to affinity features.

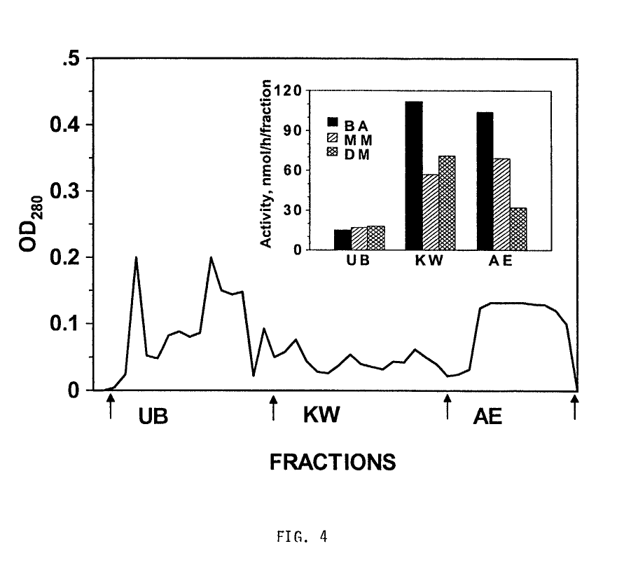

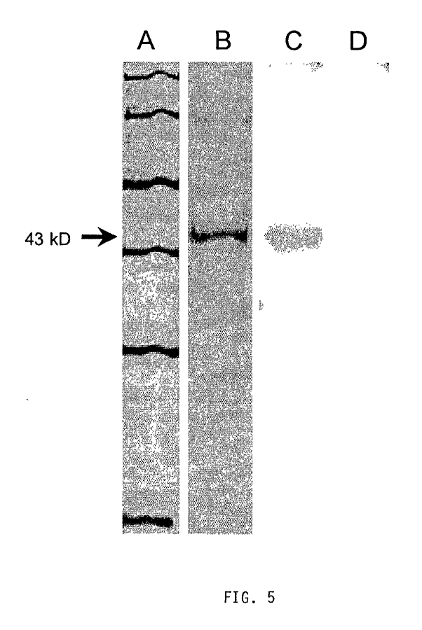

Continuous elution gel electrophoresis using a Prep Cell improved specific activities about 34 fold (Table I). From this step onward, however, the enzyme was labile and the steps needed to be performed without interruption. In the buffer system employed, the NMTase activities eluted six to nine mL after the dye front eluted. Adenosine agarose effected about a 1890-fold increase in specific activities (FIG. 4). The purified fraction methylated β-alanine, N-methyl β-alanine, and N,N-dimethyl β-alanine (Table I). However, the specific activities observed using N-methyl β-alanine and N,N-dimethyl β-alanine as substrates were less than those observed using β-alanine (Table I). The enzyme was labile in this fraction, especially for the activity against N, N-dimethyl β-alanine, with about 50% loss of activity over 12 h on ice. SDS-PAGE analysis indicated that the purified protein fraction had one major protein at about 43 kD (FIG. 5, lane B). There were minor contaminants at around 66 kD, appearing as a faint doublet in a silver-stained gel (FIG. 5, lane B). Storage of the purified protein at −80° C. resulted in the generation of a protein band at around 25 kD. The amount of this 25 kD product increased as the storage period increased.

When a partially purified protein fraction was subjected to photoaffinity labeling with S-adenosyl-L-[methyl-³H]Met, the 43 kD protein was labeled (FIG. 5, lane C). When S-adenosyl-L-homocysteine (AdoHCy) at 217 μM was added prior to crosslinking, the photoaffinity labeling was completely inhibited (FIG. 5, lane D). Experiments showed that the 43 kD affinity-labeled subunit was degrading during storage producing a labeled band about 25 kD size.

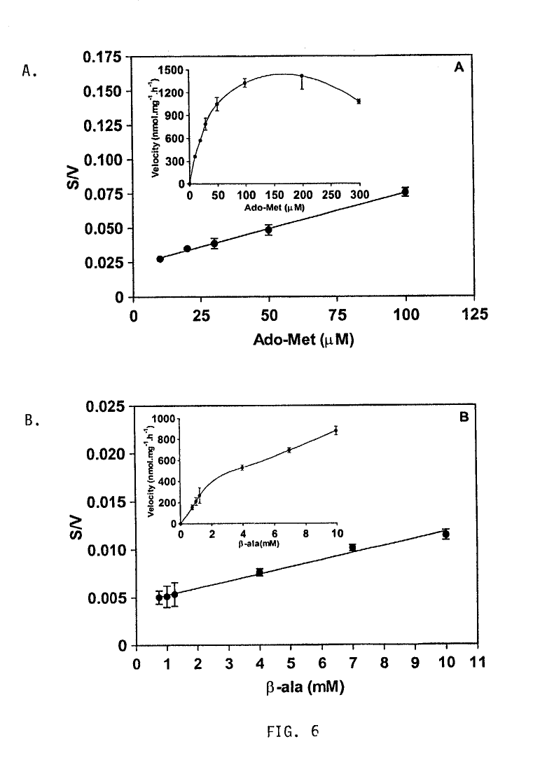

The reactions catalyzed by the NMTase exhibited Michaelis-Menten kinetics with respect to its substrate saturation response. The response for varying Ado-Met and β-alanine are shown in FIG. 6. Similar plots for N-methyl β-alanine and N,N-dimethyl β-alanine were employed to derive the kinetic parameters listed in Table II. At 10 mM β-alanine, Ado-Met exhibited substrate inhibition above 200 μM (FIG. 6A). Apparent Km for Ado-Met was 45 μM. Apparent Km for the methyl acceptor substrates determined at 100 μM Ado-Met were around 5 mM (Table II). The catalytic efficiency, Vₘₐₓ/Kₘ values were comparable for the three methyl acceptors (Table II). AdoHCy was highly inhibitory to the NMTase: 50% inhibition was achieved at 40 μM AdoHCy at 10 mM β-alanine and 100 μM Ado-Met.

Isoelectric focusing (IEF) experiments indicated a single peak of activity at a pI of 5.15. The sulfhydral reagent p-hydroxymercuribenzoic acid highly inhibited the NMTase (Table III). This inhibition was partially reversible by DTT suggesting that cysteines are involved in the active site of the NMTase.

Peptide sequences were obtained from the purified NMTase protein from L. latifolium (Sequences A-E below). Amino acids in parentheses are alternate possibilities arising from ambiguities in the sequencing.

Sequence A

(SEQ ID NO:1)

H(S/Q/A) R T E(Q) E E (L) Y R Q L G L L A G

Sequence B

(SEQ ID NO:2)

S(Q/A) L D G (A) S G (Y/E) D G F E G

Sequence C

(SEQ ID NO:3)

S (R/H/A/Q)R T E E E Y R Q L G L L A G

Sequence D

(SEQ ID NO:4)

A L L G S G Y D G F E G V K

Sequence E

(SEQ ID NO:5)

F R V I H V D Y F F P V V E F

In a BLAST search, Sequence A shared some homology with the peptide sequences of several other methyltransferases (see below), but the Sequence B did not. Sequence A showed homology to:

a. Caffeic acid O-methyltransferase-like protein of Arabidopsis thaliana emb|CAB64217.1 Sequence matched: 325 HRTEEEFIELGLSAG (SEQ ID NO:6)339;

b. Putative DNA enzyme of Eikenella corrodens gb|AAD18127.1 Sequence matched: 154 EYRQLGLLA (SEQ ID NO:7)162; and

c. O-diphenol-O-methyltransferase of Medicago sativa subsp. varia. Emb|CAB65279.1 Sequence matched 324 HRTEEQFKQLG (SEQ ID NO:8) 334.

TABLE I
Purification of an AdoMet dependent NMTase from 550 g fresh weight
leaves of L. latifolium. Fold-purification was calculated based
on specific activities measured with β-alanine.

| | | Specific Activity | | | |
|---|---|---|---|---|---|
| | Total | nmol.h.mg protein | | | |
| | protein | | N-methyl β- | N,N-dimethyl β- | Fold |
| Step | (mg) | β-alanine | alanine | alanine | purification |
| Crude | 2533.3 | 8.3 | 8.1 | 12.8 | 1 |
| 10-15% PEG | 1315.2 | 16.6 | 6.3 | 8.1 | 2 |
| Heating | 1156.2 | 15.3 | 12.0 | 13.2 | 2 |
| DEAE- | 46.8 | 47.3 | 39.9 | 40.7 | 6 |
| Fractogel | | | | | |
| Sephacryl S- | 11.3 | 46.0 | 38.0 | 32.0 | 6 |
| 200 | | | | | |
| N,N-dimethyl | 5.1 | 104.0 | 71.0 | 70.0 | 13 |
| β-alanine: | | | | | |
| Sepharose | | | | | |
| Prep Cell | 0.65 | 285.3 | 185.4 | 174.8 | 34 |
| electrophoresis | | | | | |
| Adenosine | 0.004 | 15690.0 | 9020.0 | 4195.0 | 1890 |
| Agarose | | | | | |

TABLE II
Kinetic parameters of NMTase from L. latifolium leaves.
Replots of data from substrate response experiments were
used to determine the value of the kinetic parameters.

| | | | Vmax/Km |
|---|---|---|---|
| | Apparent Km | Vmax | Catalytic |
| Substrate | (mM) | (nmol.mg.h) | efficiency |
| β-alanine | 5.28 | 1216 | 230 |
| N-methyl β-alanine | 5.68 | 1290 | 227 |
| N,N-dimethyl β-alanine | 5.87 | 1697 | 289 |
| Ado-Met | 0.045 | 1922 | 43094 |

TABLE III
Inhibition of L. latifolium NMTase by p-hydroxymercuribenzoate.
Activities are expressed as per cent total relative to control
assays containing 5 mM DTT. They were assayed, 30 min total time
in each case, against β-alanine (BA), N-methyl β-alanine
(MM) and N,N-dimethyl β-alanine (DM) as described in the methods.
Values are means and standard errors from three determinations.
pHMB = p-hydroxymercuribenzoate.

| | % Activity | % Activity | % Activity |
|---|---|---|---|
| Treatment | BA | MM | DM |
| Control, 5 mM DTT in the assays | 100 ± 4.6 | 100 ± 6.2 | 100 ± 11.4 |
| Minus DTT | 59.2 ± 1.1 | 58.7 ± 1.3 | 87.3 ± 16.0 |
| Minus DTT Plus 0.2 mM pHMB | 1.3 ± 0.7 | 2.4 ± 1.4 | 0.4 ± 0.2 |
| 0.2 mM pHMB 10 min + 5 mM | 22.6 ± 2.9 | 23.0 ± 2.9 | 55.2 ± 7.6 |
| DTT for 30 min. | | | |

### Other Embodiments

While the above specification contains many specifics, these should not be construed as limitations on the scope of the invention, but rather as examples of preferred embodiments thereof. Many other variations are possible. Therefore to apprise the public of the scope of the invention and the embodiments covered by the invention, the following claims are made.

What is claimed is:

1. A purified N-methyltransferase, the N-methyltransferase:
(a) being present in Limonium latifolium;
(b) having an isoelectric point of about 5.15; and
(c) migrating on SDS-PAGE at about 43 kilodaltons.

2. The purified N-methyltransferase of claim 1, wherein the N-methyltransferase comprises an amino acid sequence selected from the group consisting of: SEQ ID NOs: 1-5.

3. A purified protein comprising the amino acid sequence of SEQ ID NO: 1.

4. A purified protein comprising the amino acid sequence of SEQ ID NO: 2.

5. A purified protein comprising the amino acid sequence of SEQ ID NO: 3

6. A purified protein comprising the amino acid sequence of SEQ ID NO: 4.

7. A purified protein comprising the amino acid sequence of SEQ ID NO: 5.

8. A purified antibody that specifically binds an N-methyltransferase present in Limonium latifolium.

9. A purified antibody that specifically binds a polypeptide consisting of an amino acid sequence selected from the group consisting of: SEQ ID NOs: 1-5.

10. The purified antibody of claim 9, wherein the amino acid sequence is SEQ ID NO: 1.

11. The purified antibody of claim 9, wherein the amino acid sequence is SEQ ID NO: 2.

12. The purified antibody of claim 9, wherein the amino acid sequence is SEQ ID NO: 3.

13. The purified antibody of claim 9, wherein the amino acid sequence is SEQ ID NO: 4.

14. The purified antibody of claim 9, wherein the amino acid sequence is SEQ ID NO: 5.

15. A cell into which has been introduced a purified N-methyltransferase, the N-methyltransferase:
(a) being present in Limonium latifolium;
(b) having an isoelectric point of about 5.15; and
(c) migrating on SDS-PAGE at about 43 kilodaltons.

16. The cell of claim 1, wherein the N-methyltransferase comprises an amino acid sequence selected from the group consisting of: SEQ ID NOs: 1-5.

17. A cell into which has been introduced a purified protein, the protein comprising an amino acid sequence selected from the group consisting of: SEQ ID NOs: 1-5.

18. The cell of claim 17, wherein the amino acid sequence is SEQ ID NO: 1.

19. The cell of claim 17, wherein the amino acid sequence is SEQ ID NO: 2.

20. The cell of claim 17, wherein the amino acid sequence is SEQ ID NO: 3

21. The cell of claim 17, wherein the amino acid sequence is SEQ ID NO: 4.

22. The cell of claim 17, wherein the amino acid sequence is SEQ ID NO: 5.

23. The cell of claim 17, wherein the cell is a plant cell.

24. The cell of claim 23, wherein the plant cell is in a plant.


SEQUENCE LISTING (8 sequences)

SEQ ID NO: 1 - protein, 20 aa, Limonium latifolium
  UNSURE (2)..(4): (S/Q/A) AMINO ACIDS IN BRACKETS ARE ALTERNATE POSSIBILITIES INDICATING AMBIGUITY IN SEQUENCING
HSQARTEQEELYRQLGLLAG

SEQ ID NO: 2 - protein, 16 aa, Limonium latifolium
  UNSURE (2)..(3): (Q/A) AMINO ACIDS IN BRACKETS ARE ALTERNATE POSSIBILITIES INDICATING AMBIGUITY IN SEQUENCING
SQALDGASGYEDGFEG

SEQ ID NO: 3 - protein, 19 aa, Limonium latifolium
  UNSURE (2)..(5): (R/H/A/Q) AMINO ACIDS IN BRACKETS ARE ALTERNATE POSSIBILITIES INDICATING AMBIGUITY IN SEQUENCING
SRHAQRTEEEYRQLGLLAG

SEQ ID NO: 4 - protein, 14 aa, Limonium latifolium
ALLGSGYDGFEGVK

SEQ ID NO: 5 - protein, 15 aa, Limonium latifolium
FRVIHVDYFFPVVEF

SEQ ID NO: 6 - protein, 15 aa, Arabidopsis thaliana
HRTEEEFIELGLSAG

SEQ ID NO: 7 - protein, 9 aa, Eikenella corrodens
EYRQLGLLA

SEQ ID NO: 8 - protein, 11 aa, Medicago sativa
HRTEEQFKQLG